(19) United States
(12) Patent Application Publication
(10) Pub. No.: US 2025/0256120 A1
(43) Pub. Date: Aug. 14, 2025

(54) SYSTEMS AND METHODS FOR TREATING PERIODONTAL CONDITIONS WITH LIGHT

(71) Applicant: LUMENIS BE LTD., TOKNEAM (IL)

(72) Inventors: Yair Mandelstam-Manor, Yokneam Illit (IL); May Kleinman Ben Tsvi, Yokneam Illit (IL); Haya Hamza, Yokneam Illit (IL); Shelly Akazany, Yokneam Illit (IL)

(21) Appl. No.: 19/188,885

(22) Filed: Apr. 24, 2025

Related U.S. Application Data
(63) Continuation of application No. PCT/IB2023/060768, filed on Oct. 25, 2023.
(60) Provisional application No. 63/419,474, filed on Oct. 26, 2022.

(51) Int. Cl. A61N 5/06

(52) U.S. Cl. CPC A61N 5/0603; A61N 5/062; A61N 2005/0606; A61N 2005/0644; A61N 2005/0667

(57) ABSTRACT

Devices and methods for treating a periodontal condition are provided; the devises comprising an Intense Pulsed Light (IPL) source configured for generating treatment light adapted for the treatment of the periodontal condition, a probe configured for delivering the treatment light to the diseased body part or in vicinity thereof, and a control unit configured for controlling the IPL source and/or the probe and adapting treatment parameters to the periodontal condition.

### RELATED APPLICATIONS

This application is a continuation application of PCT Application No. PCT/IB 2023/060768 filed 25 Oct. 2023, which claims priority to U.S. Provisional Application No. 63/419,474 filed 26 Oct. 2022, both entitled “SYSTEMS AND METHODS FOR TREATING PERIODONTAL CONDITIONS WITH LIGHT” to which applications priority is hereby claimed and the entire contents of both are herein incorporated by reference.

### TECHNOLOGICAL FIELD

The present invention is in the medical field and relates specifically to the dental field, and more specifically to methods and devices for treating dental conditions/diseases with light.

### BACKGROUND

Periodontal diseases, such as Gingivitis and Periodontitis as well as inflammatory conditions around dental implants, including Peri-implant mucositis and Peri-implantitis, are chronic inflammatory diseases primarily resulting from interactions between the dental plaque (a biofilm of bacteria) and the immune system of the host. Secondary etiology may be related to genetic factors, systemic medical conditions, and environmental factors.

In Gingivitis, for example, the disease starts at the gingival margin and presents with swelling, bleeding, redness and discomfort. With progression of the disease, epithelial tissue, connective tissue and bone may be damaged, resulting in loss of teeth and dental implants.

Currently, an effective way to prevent periodontal diseases is through self-performed oral hygiene. Severe cases might require surgical interference. Anti-inflammatory drugs can also be helpful, however adverse events and lack of compliance may limit their efficacy.

Light-based therapy can be useful for treating some medical conditions. Intense Pulsed Light (IPL) is a light-based technology in which high-powered pulses of broad-spectrum light (wavelengths between 400 and 1200 nm) are emitted in brief pulses. IPL has been shown to be a safe and efficient method for treating inflammatory skin diseases such as Erythema of Rosacea and Acne, and it has been shown that IPL can be also useful for treating inflammatory ocular diseases such as blepharitis and dry eye disease.

### GENERAL DESCRIPTION

The present invention provides a novel technique for treating periodontal diseases with Intense Pulsed Light (IPL). Systems and methods for treating periodontal diseases utilizing IPL are described.

The anti-inflammatory activity of IPL can involve, inter alia, activation of mitochondria, increase in ATP production and boost in cellular activity. In cells which secrete anti-inflammatory agents such increase in cellular activity can result in overall reduction of inflammation. In addition, IPL can be useful for reducing the bacterial load on gingiva, the primary source of inflammation. In cells which generate oxygen radicals and reactive oxidate species, like Neutrophils, increases in their cellular activity can result in an increase in their anti-microbial activities.

In one aspect, there is provided a system for treating a periodontal condition, such as a periodontal disease and/or periodontal inflammation, with IPL, the system comprising an IPL source configured for generating light adapted for the treatment of the periodontal condition/disease, a probe/handpiece configured for delivering the light to the diseased body part/region/tissue or in vicinity thereof, and a control unit configured for controlling the IPL source and/or the handpiece and adapting the treatment parameters to the treated body part/region/tissue and the periodontal condition.

In some embodiments, the system comprises an enclosure housing said IPL source, said probe and said control unit.

In some embodiments, the system comprises a first enclosure housing said IPL source and at least a part of said control unit, and a second enclosure housing said probe.

In some embodiments, the system comprises a first enclosure housing said IPL source, a second enclosure housing said probe and a third enclosure housing said control unit.

In some embodiments, the system further comprising one or more optical filters configured to be positioned in optical path of the treatment light to define one or more of intensity and wavelength range of the treatment light.

In some embodiments, the treatment light is in a wavelength range between 400-1200 nm. In some embodiments, the treatment light is in a wavelength range between 590-1200 nm.

In some embodiments, the probe comprises a main body configured to be grasped by a user's hand, and a tip configured to be brought in proximity to or in contact with the body part. The tip may be disposable. The system may comprise a plurality of disposable tips enabling conformation to different body parts, and/or enabling reaching hard-to-reach body parts.

In some embodiments, the tip defines a treatment surface having a predetermined positioning with respect to the main body of the probe.

In some embodiments, the tip is configured to provide the treatment surface having an adjustable positioning in one or more axes with respect to the main body of the probe.

In some embodiments, the probe defines an adjustable irradiation angle of the IPL treatment light, enabling adjusting the treatment to different body parts.

In some embodiments, the probe and the treatment light are adapted for extra-oral treatment of the periodontal condition.

In some embodiments, the probe and the treatment light are adapted for intra-oral treatment of the periodontal condition.

In some embodiments, the control unit enables defining one or more of the following treatment parameters: fluence/intensity of the IPL light, duration of the IPL pulse, number of the IPL pulses, interval between the IPL pulses, the IPL pulse shape.

In some embodiments, the periodontal condition includes one or more of the following: Gingivitis, Periodontitis, Peri-implant mucositis, Peri-implantitis and inflammatory conditions around dental implants.

In another aspect, there is provided a method for treating a periodontal condition, comprising:
providing an IPL source configured and operable to generate IPL treatment signal at least in a wavelength range suitable for treating a periodontal condition;
providing a control unit configured for defining parameters of treatment of the periodontal condition and controlling the IPL source;
providing a probe for delivering the IPL signal to a body part that is intended for treatment; and
bringing the probe in proximity or in contact with the body part and activating the IPL source according to the treatment parameters, and applying the treatment to treat the periodontal condition.

In some embodiments, the method comprises providing and positioning one or more optical filters in optical path of the treatment signal/light to define one or more of intensity and wavelength range of the treatment light.

In some embodiments, the method comprises generating the treatment light in a wavelength range between 400-1200 nm.

In some embodiments, the method comprises generating the treatment light in a wavelength range between 590-1200 nm.

In some embodiments, the method comprises defining one or more of the following treatment parameters, by the control unit: intensity of the IPL light, duration of the IPL pulse, number of the IPL pulses, interval between the IPL pulses, the IPL pulse shape.

In some embodiments, the method comprises exposing the treated body part/region to the IPL indirectly through one or more mediums. The medium can be a medium/substance that enhances one or more aspects of the treatment, such as coupling the IPL to the treated body part/region or to a vicinity thereof.

In some embodiments, the method comprises providing a substance between the probe and the body part, the substance including a portion that is activated by the IPL energy to further enhance the treatment of the periodontal condition.

In some embodiments, the method includes indirect irradiation of the treated body part/region, e.g. extra-orally.

In some embodiments, the method includes local/direct irradiation of the treated body part/region, e.g. intra-orally.

In some embodiments, the method is adapted to treat one or more of the following periodontal conditions: Gingivitis, Periodontitis, including Peri-implant mucositis, Peri-implantitis and other inflammatory conditions around dental implants.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates a non-limiting exemplary embodiment of a system for treating a periodontal condition, according to the invention;

FIGS. 2A-2C illustrate non-limiting exemplary embodiments of probes of the system, according to the invention;

FIG. 3 illustrates a non-limiting exemplary embodiment of a method for treating a periodontal condition, according to the invention; and

FIGS. 4A-4C illustrate non-limiting examples of treating different periodontal conditions, utilizing the systems and methods of the invention.

### DETAILED DESCRIPTION

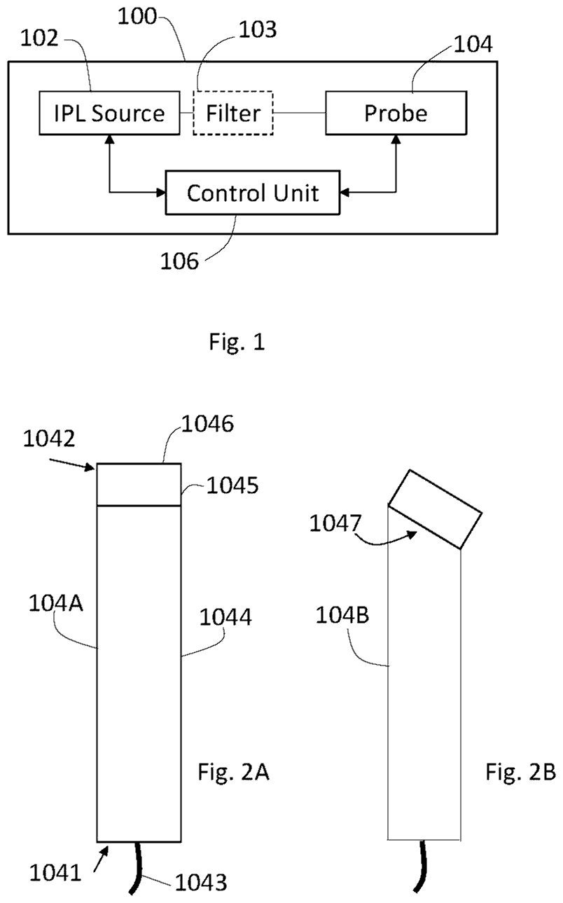

Reference is made to FIG. 1 illustrating a non-limiting example of a system 100, for treating a periodontal condition with light, specifically IPL. The system 100 includes an IPL source 102, one or more optional optical filters 103, a probe 104 and a control unit 106.

The IPL source 102, either on its own or when accompanied with the one or more optical filters, is configured for generating light in a wavelength range suitable for treating a periodontal condition, including a periodontal inflammation such as Gingivitis. The IPL source can be a X enon lamp, for example, configured for generating light in the suitable wavelength range, and can be used, in some embodiments, with various optical filters 103 to adjust and optimize the output light wavelength range per periodontal condition treatment. In some embodiments, the suitable output wavelength range is between 400-1200 nm. In some embodiments, the suitable output wavelength range is between 590-1200 nm. In some embodiments, the IPL source 102 and/or optical filter 103 can be an integral part of the probe 104. In some embodiments, the optical filter 103 can be an integral part of the IPL source 102.

The probe 104 is configured for delivering the light to the treated body part/region or in vicinity thereof to apply the treatment. In some embodiments, the probe 104 is configured to be held in hand (a handpiece). In some embodiments, the probe can be manipulated by machinery, manually or autonomously, e.g. being mounted on a robotic mechanism. The probe is configured and/or shaped for approaching the oral cavity externally or internally, to deliver the treating light to the treated body part/region. When manually operated, the probe/handpiece may include a push button that the user can use to trigger a pulse from the IPL source connected to the proximal part of the probe. The distal part of the probe/handpiece (i.e. the part farther from the operator) may include a lightguide through which the light energy is transmitted.

The control unit 106 is configured to control the IPL source and/or the probe. In some embodiments, the control unit includes a suitable user interface, such as a suitable Graphical User Interface (GUI) that enables the user to control the different functions of the system. In some embodiments, as mentioned above, one or more optical filters 103 can be placed at the output of the IPL source 102 to obtain or optimize the desired wavelength or wavelength range. This may be done, inter alia, to tailor the treatment according to the degree of pigmentation of the tissue of interest and the specific condition to be treated, and/or to control the depth of IPL penetration into the tissue. In some embodiments, a dynamic filter mechanism (not specifically shown) can be used in addition to or instead of the filter(s) 103, and which can be controlled by the control unit 106 to control the wavelength or wavelength range. In some embodiments, the control unit controls the fluence of the IPL signal, the number of sub-pulses in a light pulse, the duration of each sub-pulse, the shape of each sub-pulse, and the time intervals between the sub-pulses. Accordingly, a specific pulse sequence is adapted to each treatment and each individual.

In some embodiments, the whole system 100 can be configured as a single handpiece that includes the IPL source, the probe, the filter, and the control unit. This can be the configuration of a home-use device. In some embodiments, the system is distributed between two or three separate housings. For example, the control unit and the probe can be enclosed in one housing and the IPL source in another housing. In another example, the control unit, the probe, and the IPL source are contained in three separate housings. In some embodiments, the control unit can be distributed between a first housing that includes the IPL source with a first part of the control unit, and a second housing that includes the probe with a second part of the control unit. When the probe and the IPL source are located in separate housings, a cord including at least an optical signal carrier, such as an optical fiber, is used to connect between the housings. In some embodiments, the probe has one or more buttons that can be activated by the user to activate a treatment. In one non-limiting example, a trigger button is connected to the control unit via a wireless connection (e.g. Wi-Fi or Bluetooth).

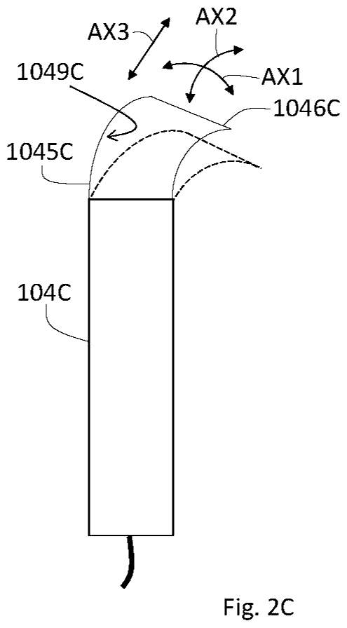

Reference is made to FIGS. 2A and 2B illustrating non-limiting examples of the probe/handpiece of the system of the invention. As shown in FIG. 2A, the probe/handpiece 104A includes a proximal side 1041 and a distal side 1042. A flexible cord 1043 is connected to the probe at the proximal side and configured to carry the IPL signal from the IPL source 102 to the probe 104A. The probe includes a main body 1044 configured for being grasped with the user's hand. The main body can be ergonomic facilitating grasping. Also, the main body includes optical elements that enable carrying the IPL signal between the proximal and distal sides. At the distal side, the probe includes a surface 1046 configured to be brought in proximity or into contact with the body part/region/tissue to be irradiated with the IPL energy, and it can have certain optical transmittance properties, e.g. it can function as an optical filter enabling transmission/blocking of specific wavelengths/wavelength ranges or as lightguide. In some embodiments, the surface 1046 can be made from a biocompatible material, and can also be a flexible material, such as silicon, that can adapt to the contours of the body part/region/tissue it contacts/engages. In some embodiments, the probe/handpiece includes or is attachable to a tip 1045 at the distal side, and the surface 1046 forms the distal end of the tip. The tip 1045 can be replaceable/disposable in order to ensure sterility of the part that is in contact with the body part. The tip can also come in different sizes/shapes to suit different patients and/or different treated body parts/regions/tissues. For example, the tip 1045 can be adjusted to conform to the oral cavity in cases the probe is inserted to the oral cavity for internal mouth treatment.

In FIG. 2B, the shown probe 104B can have all of the features described above in probe 104A, and also the probe 104B has an irradiation path/route/angle 1047, which can be different from the longitudinal axis of the probe's main body, to facilitate reaching the treated body part/region/tissue. In some embodiments, the irradiation path/route/angle is adjustable. In some embodiments, as shown in the figure, the adjustable irradiation path/route/angle is achieved by an angle between the main body and the tip. In some embodiments, the adjustable path/route/angle is achieved by optical elements such as mirrors, located either in the main body or in the tip or in both. In some embodiments, the irradiation angle can be achieved using a transmittance lightguide rod tip that can come in different angles, and the user can replace the tip per need.

Reference is made to FIG. 2C illustrating a non-limiting example of the probe/handpiece 104C having a tip 1045C with adjustable irradiation path/angle. As mentioned above, the tip 1045C forms an adjustable lightguide to carry the treatment light to the treatment surface 1046C. In some embodiments, the tip's inner side/walls 1049C are coated with a reflective material configured for reflecting the light rays passing through towards the surface 1046C and/or at least minimizing absorbance of the light energy passing through. The tip can be made from a flexible material that enables adjusting the orientation of the treatment surface 1046C, in one or more directions. In one example, the tip enables adjusting the treatment surface along one axis A X 1 (as illustrated by the dashed lines). In another example, the tip enables adjusting the treatment surface along a first axis A X 1 and a second axis A X 2 (orthogonal to A X 1, i.e. in the direction into and out of the drawing surface), thereby enabling to adjust the spatial orientation/inclination of the treatment surface in three dimensions. In another example, the tip enables adjusting the treatment surface along a first axis A X 1 and a second axis A X 2 and a third axis A X 3 that enables extending or retracting the treatment surface with respect to the main body of the probe. In some embodiments, the tip can be in the form of a gooseneck with flexibility to all directions, the gooseneck would be a hollow tube forming a lightguide and the light is transmitted inside the tube and possibly reflected from the inside wall that is coated with a reflecting coating. As long as the angle of the meeting between the light ray and the tube's wall isn't above a critical angle, the light ray continues to propagate forward towards the treatment surface 1046C. For example, the critical angle condition can be met by limiting the tip wall's (gooseneck's) bending capability.

In some embodiments, the treatment light is transmitted through the probe by an optical fiber or fiber bundle. In one example, the optical fiber/fiber bundle can be integrated within the tip into a flexible jacket (such as a gooseneck jacket) which enables rotating the treatment surface and aligning it to the direction needed for easy access to various parts of the internal side of the mouth. The treatment light is transmitted fully to the tip 1046C and to the treated body part or organ with minimum loss of efficiency.

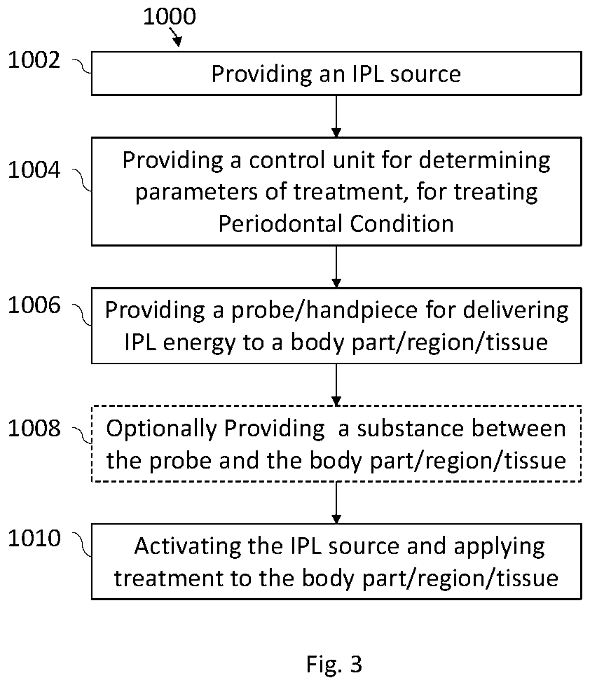

Reference is made to FIG. 3 illustrating, by way of a block diagram, a non-limiting method 1000 for treating a periodontal condition with IPL.

At step 1002, the method includes providing an IPL source, such as IPL source 102, solely or equipped with optical filter 103, configured and operable to generate IPL signal at least in a wavelength range suitable for treating a periodontal condition. As explained above, the IPL source 102 can have a wide wavelength range output and optical filters 103 can be used at the source's output to tailor the wavelength(s) to the required treatment.

At step 1004, the method includes providing a control unit, such as the control unit 106, for determining parameters of treatment of the periodontal condition and controlling the IPL source. As described above, the parameters include parameters of the IPL signal, such as wavelength and intensity/fluence, and parameters relating to the protocol of treatment, such as temporal parameters, the number of pulses or sub-pulses, pulse shape, pulse duration, and interval between pulses.

At step 1006, the method includes providing a probe/handpiece for delivering the IPL signal/energy to a body part/region/tissue that is intended for treatment.

At optional step 1008, the method includes providing a substance between the probe and the body part/region/tissue. The substance can be smeared on the body part/region/tissue. The substance can be a gel. The substance can aid in coupling of the probe/IPL signal to the body part/region/tissue. The substance can be configured to enhance the effect of the IPL energy. The substance can include a portion/constituent that is activated by the IPL energy to further enhance the treatment of the periodontal condition.

At step 1010, the probe is brought in proximity/engaged with the body part/region/tissue and the IPL source is activated according to the treatment protocol/parameters, and the treatment is applied to treat the periodontal condition.

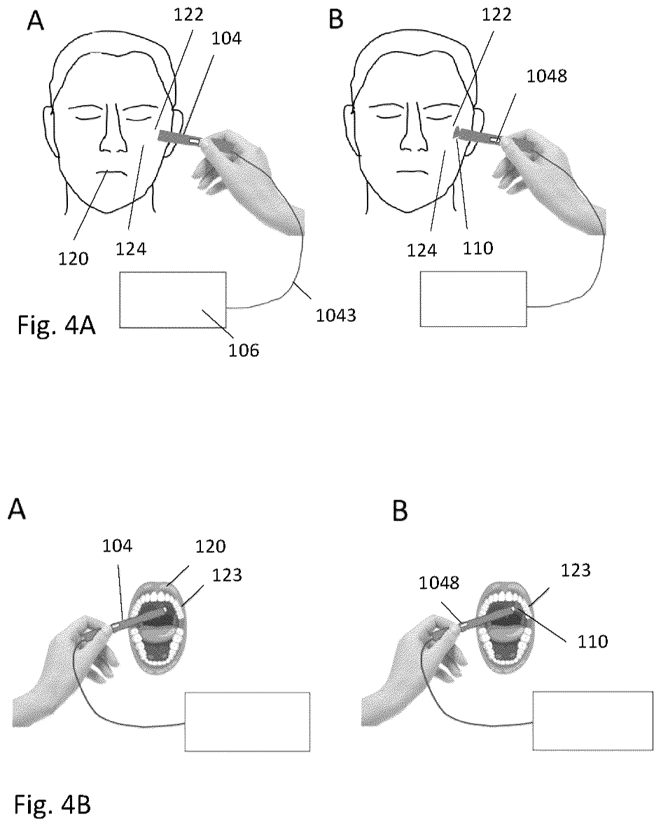

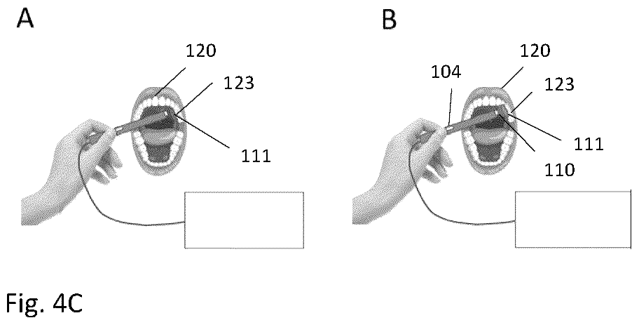

Referring to FIGS. 4A to 4C, there are illustrated non-limiting examples for treating various periodontal conditions.

In a first example, shown in FIG. 4A, IPL is administered extra-orally, on the skin compartment closest to the tissue/site of interest. For example, if the site of inflammation involves the left maxillary molars, IPL is administered on the skin of the left malar region (the cheek). In this example, as a preparation step before treatment the skin may be covered with a layer of coupling gel. As shown in FIG. 4AA, the hand of the user holds an IPL probe/handpiece 104 which is connected through a fiber or cable 1043 to an IPL control unit/console 106. The patient has his mouth 120 closed. The site of inflammation 122 on the gingiva is covered by the malar region (the cheek) 124, hence is not seen in this figure. In FIG. 4A B, the user presses a trigger button 1048, and a pulse of light 110 is emitted indirectly on the site of inflammation 122, through the skin of the malar region 124.

In a second example, shown in FIG. 4B, IPL is administered intra-orally, directly on the site of inflammation on the gingiva. In this example, the tissue of interest may or may not be covered with coupling gel. In FIG. 4BA, the hand of the user approaches an

IPL handpiece 104 to the site of inflammation 123 within the mouth 120 of the patient. In FIG. 4BB, the user presses on the trigger button 1048 and a pulse of light 110 is emitted directly onto the site of interest.

In a third example, shown in FIG. 4C, before IPL administration a soft substance 111 that activates upon exposure to light is smeared on the site of inflammation 123. For example, this substance could be Toluidine Blue, a material that is routinely used in dentistry for coloring the gingiva. During IPL administration, when the user presses on the trigger button, a pulse of light 110 is emitted, and the soft substance 111 becomes activated at the site of inflammation.

In a fourth example (not specifically shown), IPL is used in the same way with the third example, except that the soft substance 111 is injected in pockets between teeth and gingiva.

1. A system for treating a periodontal condition, comprising:
an Intense Pulsed Light (IPL) source configured for generating treatment light adapted for the treatment of the periodontal condition,
a probe configured for delivering the treatment light to the diseased body part or in vicinity thereof, and
a control unit configured for controlling the IPL source and/or the probe and adapting treatment parameters to the periodontal condition.

2. The system as claimed in claim 1, comprising an enclosure housing said IPL source, said probe and said control unit.

3. The system as claimed in claim 1, comprising a first enclosure housing said IPL source and at least a part of said control unit, and a second enclosure housing said probe.

4. The system as claimed in claim 1, comprising a first enclosure housing said IPL source, a second enclosure housing said probe and a third enclosure housing said control unit.

5. The system as claimed in claim 1, further comprising one or more optical filters configured to be positioned in optical path of the treatment light to define one or more of intensity and wavelength range of the treatment light.

6. The system as claimed in claim 1, wherein said treatment light is in a wavelength range between 400-1200 nm.

7. The system as claimed in claim 1, wherein said treatment light is in a wavelength range between 590-1200 nm.

8. The system as claimed in claim 1, wherein said probe comprises a main body configured to be grasped by a user's hand, and a tip configured to be brought in proximity to or in contact with the body part.

9. The system as claimed in claim 8, wherein said tip is disposable.

10. The system as claimed in claim 8, wherein said system comprises a plurality of disposable tips enabling conformation to different body parts.

11. The system as claimed in claim 8, wherein said tip defines a treatment surface having a predetermined positioning with respect to the main body of the probe.

12. The system as claimed in claim 11, wherein said tip is configured to have an adjustable positioning in one or more axes with respect to the main body of the probe.

13. The system as claimed in claim 1, wherein the probe and the treatment light are adapted for extra-oral treatment of the periodontal condition.

14. The system as claimed in claim 1, wherein the probe and the treatment light are adapted for intra-oral treatment of the periodontal condition.

15. The system as claimed in claim 1, wherein said control unit enables defining one or more of the following treatment parameters: intensity of the IPL light, duration of the IPL pulse, number of the IPL pulses, interval between the IPL pulses, the IPL pulse shape.

16. The system as claimed in claim 1, wherein said periodontal condition includes one or more of the following: Gingivitis, Periodontitis, Peri-implant mucositis, Peri-implantitis and inflammatory conditions around dental implants.

17. The system as claimed in claim 1, further comprising a substance configured to be positioned between the probe and the body part, the substance being characterized in one or more of the following:
for aiding in coupling between the probe and the body part, and/or
at least a portion of the substance is activated by the treatment light to further enhance the treatment of the periodontal condition.

18. A method for treating a periodontal condition, comprising:
providing an IPL source configured and operable to generate IPL treatment signal at least in a range suitable for treating a periodontal condition;
providing a control unit configured for defining parameters of treatment of the periodontal condition and controlling the IPL source;
providing a probe for delivering the IPL signal to a body part that is intended for treatment; and
bringing the probe in proximity or in contact with the body part and activating the IPL source according to the treatment parameters and applying the treatment to treat the periodontal condition.

19. The method as claimed in claim 18, comprising providing a substance between the probe and the body part, for aiding in coupling between the probe and the body part, the substance including a portion that is activated by the IPL treatment signal to further enhance the treatment of the periodontal condition.
